(19) United States
(12) Patent Application Publication
(10) Pub. No.: US 2011/0028831 A1
(43) Pub. Date: Feb. 3, 2011

(54) PERMANENTLY VISIBLE IMPLANTABLE FIDUCIARY TISSUE MARKER

(76) Inventor: James P. Kent, Minneapolis, MN (US)

Correspondence Address: WESTMAN CHAMPLIN & KELLY, P.A., SUITE 1400, 900 SECOND AVENUE SOUTH, MINNEAPOLIS, MN, 55402, US

(21) Appl. No.: 12/512,523

(22) Filed: Jul. 30, 2009

(51) Int. Cl. A61B 5/05

(52) U.S. Cl. 600/426

(57) ABSTRACT

A fiduciary marker includes a hollow cylindrical body comprised of a bio-compatible polymer and including a first end, a second end, an inner surface extending between the first end to the second end and an outer surface extending between the first end to the second end. A plurality of apertures are formed in the body and extend from the inner surface to the outer surface.

### BACKGROUND

Fiduciary markers are devices used for marking points of reference in tissue for later medical imaging. Certain medical conditions, including various types of cancer, are increasingly being diagnosed and treated using minimally invasive medical techniques. Such techniques typically involve the use of clinical imaging methods that allow the physician to visualize interior tissue of a patient's body without the need to make excessive incisions. The fiduciary marker can be implanted into tissue of a patient during surgical procedures, such as biopsies.

A fiduciary marker can include a solid object that is implantable into tissue by itself, the object can be surrounded by a gelatinous matrix to temporarily increase visibility or the object can incorporate a contrast agent. These gelatinous matrices and contrast agents are used to improve visibility of an image in certain modalities, but such gels are not permanent. Because each imaging modality has different needs, the same fiduciary marker can fail to be clearly visible in multiple different imaging modalities as well as can fade over time.

In particular, while a fiduciary tissue marker can appear well in an x-ray image, the same marker can appear as a void or dark artifact in a magnetic resonance image (MRI). This can be particularly problematic in some contexts. For example, heterogeneous breast tissue produces many dark artifacts under MR imaging that render voids produced by a marker difficult to identify and distinguish from naturally occurring dark artifacts. In addition, some markers produce large susceptibility artifacts under MR imaging, thereby distorting images in both MRI and spectroscopic modalities. With the increasing use of MRI and ultrasound techniques in the treatment of breast cancer, a permanent tissue marker having improved visibility in a variety of different imaging modalities is important.

### SUMMARY

A fiduciary marker includes a hollow cylindrical body comprised of a bio-compatible polymer and including a first end, a second end, an inner surface extending between the first end to the second end and an outer surface extending between the first end and the second end. A plurality of apertures are formed in the body and extend from the inner surface to the outer surface. The fiduciary marker can include a metallic bar extending at least within the interior of the hollow cylindrical body along the inner surface at least partially between the first and the second end. The bio-compatible polymer of the hollow cylindrical body can be compounded with a radiopaque material.

### BRIEF DESCRIPTION OF THE DRAWINGS

FIG. 1 illustrates a perspective view of a fiduciary marker under one embodiment.

FIG. 2 illustrates an end view of the fiduciary marker illustrated in FIG. 1.

FIG. 3 illustrates an ultrasound image of the fiduciary marker illustrated in FIG. 1.

FIG. 4 illustrates a perspective view of a fiduciary marker under another embodiment.

FIG. 5 illustrates an end view of the fiduciary marker illustrated in FIG. 4.

FIG. 6 illustrates a perspective view of a fiduciary marker under yet another embodiment.

FIG. 7 illustrates an end view of the fiduciary marker illustrated in FIG. 6.

### DETAILED DESCRIPTION OF ILLUSTRATIVE EMBODIMENTS

Embodiments described are directed to a fiduciary marker for permanent visible implantation into various tissues of a body. Embodiments of the fiduciary marker comprise a material type and structural features that allow the marker to be clearly visible—as long as the marker remains implanted in tissue—under a variety of different medical imaging modalities, such as radiographs (x-ray, mammography, fluoroscopy, kV and computed tomography (CT)), magnetic resonance imaging (MRI) and ultrasonography imaging (ultrasound). The embodiments of the fiduciary marker described can be placed in soft tissue during open percutaneous, or endoscopic procedures to mark a surgical location for medical imaging. Such markers enable radiologists to localize the site of surgery in subsequent imaging studies or to facilitate image registration during image-guided therapeutic procedures. In this way, markers can serve as landmarks that provide a frame of reference for the radiologist.

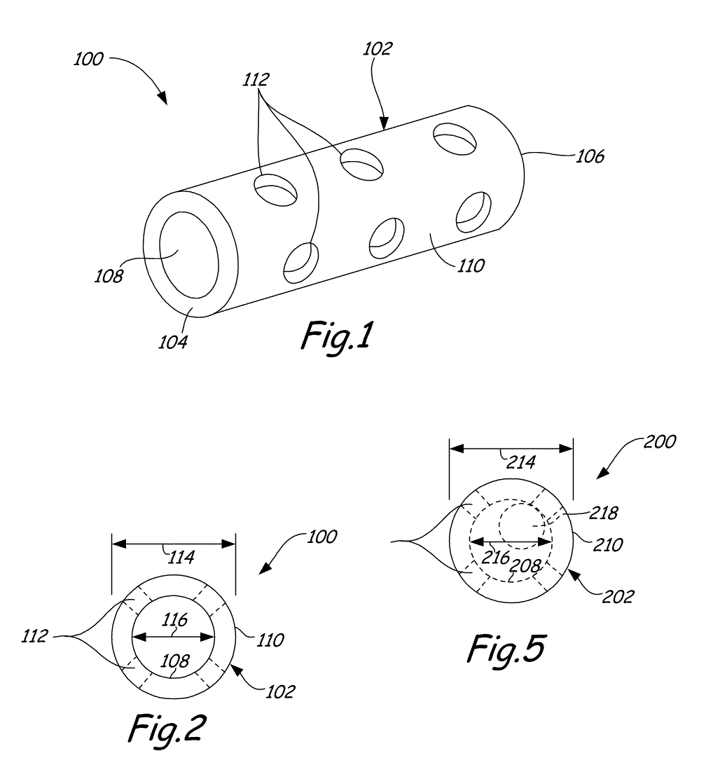

FIG. 1 illustrates a perspective view of a fiduciary marker 100 under one embodiment, while FIG. 2 illustrates an end view of fiduciary marker 100. As illustrated, fiduciary marker 100 includes a hollow cylindrical body 102 having a first end 104, a second end 106, an inner surface 108 and an outer surface 110. Inner surface 108 and outer surface 110 extend between first end 104 and second end 106. The distance between first end 104 and second end 106 can range between approximately 2 and 8 mm. In one embodiment, the distance between first end 104 and second end 106 is approximately 3 mm. In another embodiment, the distance between first end 104 and second end 106 is approximately 5 mm.

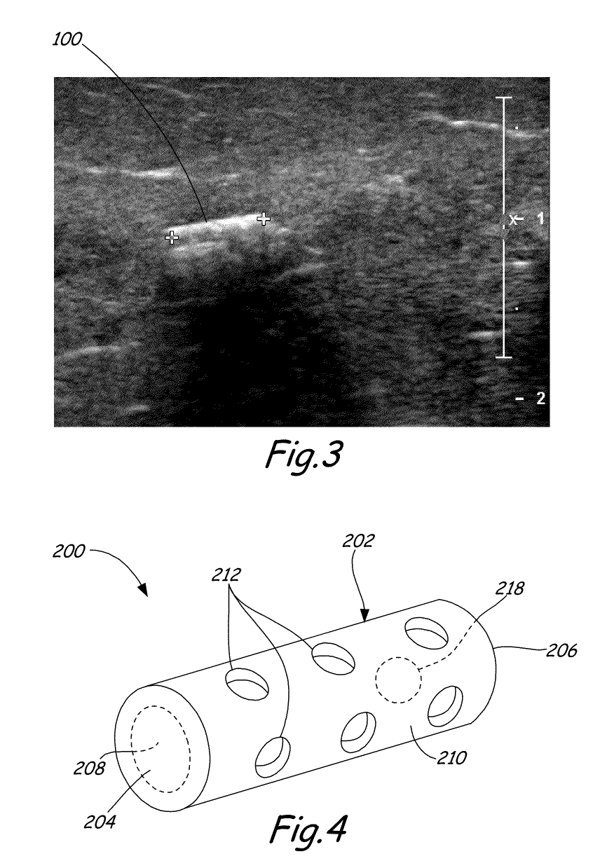

First end 104 and second end 106 of hollow cylindrical body 102 include an outer diameter 114 defined by outer surface 110 and an inner diameter 116 defined by inner surface 108. In one embodiment, outer diameter is approximately 1.5 mm and inner diameter is approximately 1 mm. As illustrated in FIGS. 1 and 2, first and second ends 104 and 106 provide hollow cylindrical body 102 with a tubular configuration such that the first and second ends 104 and 106 are open to the hollow interior of body 102. This hollow tubular shape has several advantages when imaged under different types of imaging modalities. For example, the shape of fiduciary marker 100 allows for optical acoustic reverberations, which enhances visibility under ultrasound imaging as illustrated in the ultrasound image in FIG. 3.

Body 102 includes a plurality of apertures 112. As illustrated in both FIGS. 1 and 2 and more clearly by the dashed lines in FIG. 2, apertures 112 extend from inner surface 108 to outer surface 110. In one embodiment, the diameter of each aperture 112 is approximately 0.5 mm. The plurality of apertures 112 included in hollow cylindrical body 102 are formed in a plurality of rows that span from first end 104 to second end 106. However, it should be realized that apertures can be formed in hollow cylindrical body 102 between outer surface 110 and inner surface 108 in any fashion, including a random arrangement or an unevenly spaced apart fashion.

In the FIG. 1 embodiment, the rows are evenly spaced about hollow cylindrical body 102 and include four rows of three apertures 112. In one embodiment, the distance between the edges of each aperture is approximately 1 mm. In this embodiment, the edges of each aperture located closest to the first end 104 or second end 106 are spaced from the first end or the second end by approximately 0.75 mm. In another embodiment, the distance between the edges of each aperture can be approximately 0.5 mm. In this alternative embodiment, the edges of each aperture located closest to the first end 104 or second end 106 are spaced from the first end or the second end by 0.25 mm. The combination of a hollow tubular body 102 with apertures 112 provides a structure that anchors the fiduciary marker 100 in tissue while preventing migration. In addition, these structural elements allow tissue to form in and around for better visibility in various imaging modalities.

FIG. 4 illustrates a perspective view of a fiduciary marker 200 under another embodiment, while FIG. 5 illustrates an end view of fiduciary marker 200. Like fiduciary marker 100 in FIGS. 1 and 2, fiduciary marker 200 includes a hollow cylindrical body 202 having a first end 204, a second end 206, an inner surface 208 and an outer surface 210. Inner surface 208 and outer surface 210 extend between first end 204 to second end 206.

Unlike fiduciary marker 100, first end 204 and second end 206 of fiduciary marker 200 encloses the interior of hollow cylindrical body 202. Although body 202 includes an outer diameter 214 defined by outer surface 210 and an inner diameter 216 defined by inner surface 208 as in body 102, ends 204 and 206 enclose the hollow interior of body 202.

Like body 102, body 202 includes a plurality of apertures 212 formed about body 202. As illustrated in both FIGS. 4 and 5 and more clearly by the dashed lines in FIG. 5, apertures 212 extend from inner surface 208 to outer surface 210. Unlike fiduciary marker 100, fiduciary marker 200 includes a bead enclosed in hollow cylindrical body 202. In the embodiment illustrated in FIGS. 4 and 5, bead 218 is a spherical bead having a diameter that is greater than the diameter of each aperture 212. For example, spherical bead 218 can have a diameter greater than approximately 0.5 mm, yet less than an inner diameter 208 of body 202 (i.e., approximately 0.51 mm to 0.98 mm). It should be realized, however, that bead 218 can have other shapes than a spherical shape as long as the bead is larger than apertures 212. Bead 218 can comprise glass (biocompatible silicate-based or ceramic-based), a metallic material, such as gold, platinum, stainless steel, titanium, or a biocompatible polymer, such as polyetherketoneketone (PEKK) or polyetheretherketone (PEEK). It should be understand that other biocompatible polymeric materials can be used.

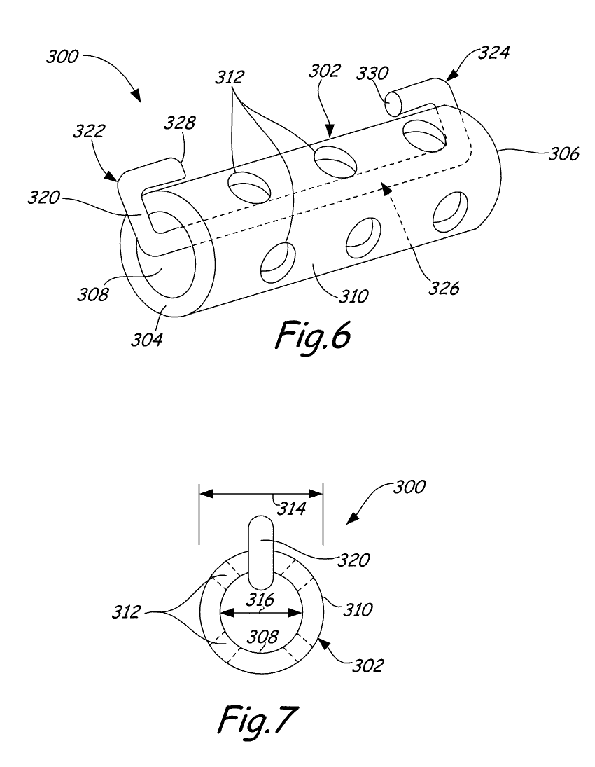

FIG. 6 illustrates a perspective view of a fiduciary marker 300 under yet another embodiment, while FIG. 7 illustrates an end view of fiduciary marker 300. Like fiduciary marker 100 and 200 in FIGS. 1 and 4, fiduciary marker 300 includes a hollow cylindrical body 302 having a first end 304, a second end 306, an inner surface 308 and an outer surface 310. Inner surface 308 and outer surface 310 extend between first end 304 to second end 306.

Like body 100 of FIG. 1, first end 304 and second end 306 of hollow cylindrical body 302 include an outer diameter 314 defined by outer surface 310 and an inner diameter 316 defined by inner surface 308. As illustrated in FIGS. 6 and 7, first and second ends 304 and 306 provide hollow cylindrical body 302 with a tubular configuration such that the first and second ends 304 and 306 are open to the hollow interior of body 302.

Like body 102 of FIG. 1 and body 202 of FIG. 4, body 302 includes a plurality of apertures 312 formed about body 302. As illustrated in both FIGS. 6 and 7 and more clearly by the dashed lines in FIG. 7, apertures 312 extend from inner surface 308 to outer surface 310. Unlike fiduciary markers 100 and 200, fiduciary marker 300 includes a bar 320 extending at least within the interior of hollow cylindrical body 302 along the inner surface 308. Although not specifically illustrated in FIG. 6, bar 320 need only extend at least partially between first end 306 and second end 308. Bar 320 has a diameter that is less than a diameter of inner diameter 308. For example, bar 320 can have a diameter ranging between 10 μm and 200 μm. Bar 320 can comprise a metallic material, such as gold, platinum, stainless steel or titanium.

Bar 320 includes a first end portion 322 and a second end portion 324 coupled to either end of a linear portion 326. First end portion 322 protrudes from first end 306 of body 302 and terminates at a first end 328. First end portion 322 wraps around body 302 such that the first end 328 terminates proximal to outer surface 310 between first end 306 and second end 308 of body 302. Second end portion 324 protrudes from second end 308 of body 302 and terminates at a second end 330. Second end portion 324 wraps around body 302 such that the second end 330 terminates proximal to outer surface 310 between first end 306 and second end 308 of body 302. Such a configuration secures bar 320 to body 302. It should be realized that bar 320 can be of other configurations and be secured to body 302 by other means.

In one embodiment, fiduciary markers 100, 200 and 300 can be made of a polymer-based material formed by injection molding. More specifically, fiduciary markers 100, 200 and 300 can be made of a polymer-based material compounded with a radiopaque material, such as a metal oxide. For example, fiduciary markers 100, 200 and 300 can be made of polyetherketoneketone (PEKK) compounded with barium sulfate. The metal oxide, such as barium sulfate, is suspended in the polymer-based material, such as PEKK, by a 10 to 30% by weight quantity to provide a high level of contrast and enhance visibility with surrounding tissue in certain types of imaging modalities, such as radiographs. In particular, barium sulfate can be compounded with a bio-compatible polymer at a 20% by weight quantity. It should be realized that other combinations of biocompatible polymer compounded with a radiopaque material, such as metal oxide, can be used. Example biocompatible polymers include polyetheretherketone (PEEK), polyalkylacrylate, polyfluoroalkylene, polyurethane, polyalkylene, polyoxyakylene, polyester, polysulphone, polycarbonate, polyacid, polyalkylene oxide ester, polyvinylchloride, silicone, polysiloxane, nylon, polyaryletherketone, polarylethersulphone, polyether imide and any copolymer which includes any of the aforementioned. Example metal oxides include Bismuth Subcarbonate, Bismuth Trioxide, Bismuth Oxychloride, Tantalum, Tungsten and Zirconium oxide.

Fiduciary markers 100, 200 and 300 are considered permanent markers because their visibility in different imaging modalities will remain constant over time. Unlike markers that include gel matrices and contrast agents, markers 100, 200 and 300 that are made of a bio-compatible polymer material will permanently hold visibility.

Although the present invention has been described with reference to preferred embodiments, workers skilled in the art will recognize that changes may be made in form and detail without departing from the spirit and scope of the invention.

What is claimed is:

1. A fiduciary marker comprising:
a hollow cylindrical body comprised of a bio-compatible polymer and including:
a first end;
a second end;
an inner surface extending between the first end to the second end;
an outer surface extending between the first end to the second end; and
a plurality of apertures formed in the body and extending from the inner surface to the outer surface.

2. The fiduciary marker of claim 1, wherein the plurality of apertures are formed in a plurality of rows about the hollow cylindrical body and extending between the first end to the second end of the body.

3. The fiduciary marker of claim 2, wherein each row comprises at least two apertures.

4. The fiduciary marker of claim 1, wherein the first and the second ends of the hollow cylindrical body comprise an outer diameter defined by the outer surface and an inner diameter defined by the inner surface such that the first end and the second end include openings to an interior of the hollow cylindrical body.

5. The fiduciary marker of claim 1, wherein the first and the second ends of the hollow cylindrical body comprise an outer diameter defined by the outer surface such that the first and the second end enclose an interior of the hollow cylindrical body.

6. The fiduciary marker of claim 1, further comprising a bead enclosed in the cylindrical hollow body.

7. The fiduciary marker of claim 6, wherein the bead is a spherical bead comprising a diameter that is greater than a diameter of the plurality of apertures formed in the body.

8. The fiduciary marker of claim 6, wherein the bead comprises a glass bead.

9. The fiduciary marker of claim 6, wherein the bead comprises a metallic bead.

10. The fiduciary marker of claim 1, further comprising a metal bar extending at least partially within the interior of the hollow cylindrical body along the inner surface at least between the first and the second end.

11. A fiduciary marker comprising:
a cylindrical body comprised of a bio-compatible polymer compounded with a radiopaque material and having a first end, a second end and an outer surface.

12. The fiduciary marker of claim 11, wherein the radiopaque material comprises a metal oxide.

13. The fiduciary marker of claim 11, wherein the cylindrical body comprises a hollow cylindrical body having a first end, a second end, an inner surface extending between the first end to the second end, an outer surface extending between the first end to the second end, and a plurality of apertures formed in the body and extending from the inner surface to the outer surface, wherein the metal oxide comprises barium sulfate.

14. The fiduciary marker of claim 13, wherein the first and the second ends of the hollow cylindrical body comprise an outer diameter defined by the outer surface and an inner diameter defined by the inner surface such that the first end and the second end include openings to an interior of the hollow cylindrical body.

15. The fiduciary marker of claim 13, wherein the first and the second ends of the hollow cylindrical body comprise an outer diameter defined by the outer surface such that the first and the second end enclose an interior of the hollow cylindrical body.

16. The fiduciary marker of claim 15, further comprising a bead enclosed in the cylindrical hollow body.

17. A fiduciary marker comprising:
a hollow cylindrical body comprised of a polymer and including:
a first end;
a second end;
an inner surface extending between the first end to the second end;
an outer surface extending between the first end to the second end;
a plurality of apertures formed in the body and extending between the inner surface to the outer surface; and
a metallic bar extending at least within the interior of the hollow cylindrical body along the inner surface at least partially between the first and the second end.

18. The fiduciary marker of claim 17, wherein the metal bar comprises titanium.

19. The fiduciary marker of claim 17, wherein the first and the second ends of the hollow cylindrical body comprise an outer diameter defined by the outer surface and an inner diameter defined by the inner surface such that the first end and the second end include openings to an interior of the hollow cylindrical body

20. The fiduciary marker of claim 19, wherein the metal bar comprises a first end portion and a second end portion coupled to a linear portion, the first end portion protrudes from the first end of the body and terminates at a first end, the second end portion protrudes from the second end of the body and terminates at a second end.
